(19) United States
(12) Patent Application Publication
(10) Pub. No.: US 2014/0200517 A1
(43) Pub. Date: Jul. 17, 2014

(54) MEDICAL LINE SECUREMENT SYSTEM

(71) Applicants: Murray Humphries, Surrey (GB); Stephen Cooper, Surrey (GB)

(72) Inventors: Murray Humphries, Surrey (GB); Stephen Cooper, Surrey (GB)

(73) Assignee: TCB-ARROW LIMITED, Surrey (GB)

(21) Appl. No.: 14/238,090

(22) PCT Filed: Aug. 10, 2012
(86) PCT No.: PCT/GB2012/000651

§ 371 Date: Feb. 10, 2014

Related U.S. Application Data
(60) Provisional application No. 61/522,354, filed on Aug. 11, 2011.

(30) Foreign Application Priority Data
Aug. 10, 2011 (GB) 1113785.8

(51) Int. Cl. A61M 25/02

(52) U.S. Cl. CPC A61M 25/02
USPC 604/180

(57) ABSTRACT

The present invention relates to a securement system for securing a medical line or device to a patient, for example, for holding a tube such as a catheter or a drip to the body of a patient. The medical treatment of patients commonly involves the use of percutaneously inserted medical lines to direct fluids directly into the bloodstream, a specific organ or an internal location within the patient, or to monitor vital functions of the patient. A health care professional commonly employs adhesive bandages, plasters or surgical tape to attach a catheter or medical line to a patient, to maintain the catheter or medical line in place with respect to the skin of the patient. To adjust the position of the device the adhesive bandage would have to be removed and replaced. This often leads to poor positioning of the tube because any adjustment causes unnecessary inconvenience and discomfort to the patient and wastes bandages. The present invention seeks to provide an improved line securement device. In particular, the present invention seeks to provide a line securement device which can adapt to various medical devices; can simply enable adjustment of position of the medical device. Specifically, the present invention seeks to provide a simple securement device that retains a medical line in a fixed position relative to an intravenous entry point and is capable of adjustment and simple release of a medical line for dressing changes, inspection and other requirements.

### FIELD OF THE INVENTION

The present invention relates to a securement system for securing a medical line or device to a patient, for example, for holding a tube such as a catheter or a drip to the body of a patient.

### BACKGROUND TO THE INVENTION

The medical treatment of patients commonly involves the use of percutaneously inserted lines to direct fluids directly into the bloodstream, a specific organ or an internal location within the patient, or to monitor vital functions of the patient. For instance, short, peripherally-inserted, intra-arteriovenous catheters are commonly used to provide medication and direct fluids directly into the bloodstream of the patient, otherwise commonly referred to as intravenous (IV) catheters. It is common in the treatment of patients to utilize the catheter in a variety of ways. For example, a directed fluid catheter typically drains from a container positioned above the patient to feed under gravity or is delivered via an infusion pump. The fluid flows through tubing and thence into an indwelling catheter. The catheter and the fluid tubing are commonly removably attached to each other by a conventional luer-lock connector.

A health care professional commonly employs adhesive bandages, plasters or surgical tape to attach a catheter or medical line to a patient, to maintain the catheter or medical line in place with respect to the skin of the patient. To adjust the position of the device the adhesive bandage would have to be removed and replaced. This often leads to poor positioning of the tube because any adjustment causes unnecessary inconvenience and discomfort to the patient and wastes bandages.

In addition, a catheterization process often requires relatively frequent disconnection between the catheter and the fluid supply tube, as well as dressing changes. For instance, intravenous catheterization is frequently maintained for several days, depending upon the condition of the patient. The tubing is generally replaced every 48 to 72 hours in order to maintain the sterility of the fluid and the free-flow of the fluid through the tubing. A health care professional must therefore frequently change the tubing and re-tape the connection. The health care professional also must frequently clean the insertion site about the indwelling catheter and change the dressings. The tape may also be applied and re-applied since the catheter—or any type of medical line—may not have been positioned correctly in the first instance.

The health care professional thus uses a great deal of valuable time applying, removing and reapplying tape. The frequent application and removal of the tape also commonly excoriates the patient's skin about the insertion site. Many patients also do not rest comfortably and worry about catheter dislodgement when they move, when only tape and a dressing secure the catheter in place. Taped intravenous catheters are also easily pulled out during a “routine” dressing change, especially by inexperienced health care providers. And if the catheter migrates too far or dislodgement occurs, the health care provider must replace the catheter, thus exacerbating the time and expense required to maintain the intravenous feed.

Several additional drawbacks result from the use of tape to stabilize the catheter. One is contamination. Health care providers often tear off small strips of tape and place them on the hand rail on the patient's bed. Clostridium and other bacteria, however, commonly exist on these surfaces and can be transferred to the patient's skin in the proximity of the insertion site. Furthermore, the area around the tape is a trap for contaminant build-up, harbouring bacteria. In addition, tape securement requires the nurse to handle the tape while wearing protective latex gloves. Tearing adhesive tape tends to produce microscopic and/or visible holes in the gloves and thus destroys glove barrier protection. Prior securement methods also have not served the patient well.

A number of catheterization systems have been developed to improve the stability of the catheter and to obviate the need for frequent application and removal of surgical tape. Prior devices, however, have generally held the connector on the IV tubing securely against the patient, rather than the catheter fitting itself.

U.S. Pat. No. 6,224,571 provides a securement device which includes a simply-structured retention mechanism that permits a portion of a catheter hub or a similar medical article to be easily anchored to a patient, without the use of tape or needles and suturing. A unitary retainer includes a base defining a locator mechanism, which mechanism interacts with a fitting of the medical device so as to position the medical device relative to the retainer. In one form, the locator mechanism includes a cradle defining an arcuate channel to receive a convex surface of the medical device, or a recess shaped to matingly receive the fitting. Whilst the retainer is attached to a flexible anchor pad that includes an adhesive bottom surface, which can be attached to the patient's skin, the catheterisation is necessarily a two-part system: an intravenous part connects with a second line via a connection system such as a leer lock.

WO2010102153 provides a device which permits a portion of a catheter or similar medical article to be easily anchored to a patient, desirably without the use of tape or needles and suture. A securement system for an elongated medical article comₚrises an anchor pad and a retainer mounted upon the anchor pad. The retainer includes a channel that has at least one abutment surface corresponding to a contact surface on the medical article and a groove configured to receive a spline of the medical article. The medical article must conform with the channel and is medical article specific.

### OBJECT TO THE INVENTION

The present invention seeks to provide an improved line securement device. In particular, the present invention seeks to provide a line securement device which can adapt to various medical devices; can simply enable adjustment of position of the medical device

Specifically, the present invention seeks to provide a simple securement device that retains a medical line in a fixed position relative to an intravenous entry point and is capable of releasing the medical line for dressing changes, inspection and other requirements. The present invention also seeks to provide a securement system that can locate and secure medical devices as well as IV lines, for example.

### SUMMARY OF THE INVENTION

In accordance with a first aspect of the present invention, there is provided a line securement device comprising a base; a securement tail and lock, wherein the base has a first face to which an adhesive is applied and a second face to which the securement tail and lock are connected; wherein the securement tail is attached to the base at a proximal end, and extend along a length to a distal end, the tail having a substantially uniform section and having a detent rack upon an engagement side of the tail, the tail extending in a first direction from the base; wherein the lock extends from the base and defines a jaw assembly, directed in said first direction from the base and a ratchet, wherein the jaw in a first, rest position, provides a width for passage of said section of said securement tail whilst the ratchet can engage with the teeth of the detent rack to allow movement of the securement tail in a first direction and to prevent movement in a direction opposite to the first direction, at least one jaw member being operable to move the jaws such as to allow passage of a section of the securement tail therethrough.

In use, the first face of the base is attached to a patient, the distal end of the securement tail can be introduced through the jaw to define a closed ring such that the securement tail can be pulled through the jaws to reduce the size of the closed ring, in a non-return fashion and wherein at least one of the jaws can be widened against a resilient bias whereby to enable removal of the tail and thus release of the line or device.

Preferably, the securement device's base element is attached to an adhesive skin contacting patch member, which in use directly anchors the system to the skin of a patient. Conveniently, the patch member has a sufficient thickness and tackiness, to either secure the medical line or device in order to inhibit relative movement between the securement device and medical line or device.

Conveniently, the jaws define between themselves a passageway of a width, which width also corresponds to a width of the securement tail. In a further preferred embodiment, the jaws are provided with finger paddles, operable to cause one or both of the jaws to move outwardly, whereby to widen the gap between the jaws. The jaws can also be provided with sloped entry portions to enable the jaws to open upon presentation of the tail against said sloped portions.

The tail, base and jaws can be formed as separate elements and can be connected together without the aid of adhesive, welding, or by use of detent means. In the alternative, the tail, base and jaws are integrally formed as a unitary piece.

During the application process, a health care professional need not contact the adhesive layers with his gloves. Use of the securement device thus does not degrade glove protection. Once secured, the securement system stabilizes a catheter or medical device and substantially prevents catheter movement and migration. Furthermore, the present invention has no need for the use of tapes which, because of the exposed adhesive that will become a site for bacterial growth, the minimization of such exposure substantially reduces the risk of an insertion site infection.

Further aspects, features, and advantages of the present invention will become apparent from the detailed descriptions of the preferred embodiments that follow.

### BRIEF DESCRIPTION OF THE FIGURES

Some preferred embodiments of the invention will now be described, by way of example, with reference to the accompanying drawings, of which:

FIG. 1 is a perspective view of a medical line securement device in accordance with a first embodiment of the present invention;

FIG. 2 is an exploded view of the medical line securement device of FIG. 1;

FIGS. 3-5 show the first embodiment in first, second and third side views;

FIGS. 6 & 7 show front and rear perspective views of one embodiment;

FIGS. 8 & 9 show the securement tail of the present invention in perspective and side views;

FIGS. 10 and 11 show the base and jaws of the first embodiment of the invention;

FIGS. 12 and 13 show an embodiment of the invention upon a bench and when attached to a patient, in use; and,

FIGS. 14a, b & c show a still further embodiment of the invention.

### DETAILED DESCRIPTION OF THE PREFERRED EMBODIMENTS

There will now be described, by way of example only, the best mode contemplated by the inventor for carrying out the present invention. In the following description, numerous specific details are set out in order to provide a complete understanding to the present invention. It will be apparent to those skilled in the art, that the present invention may be put into practice with variations of the specific.

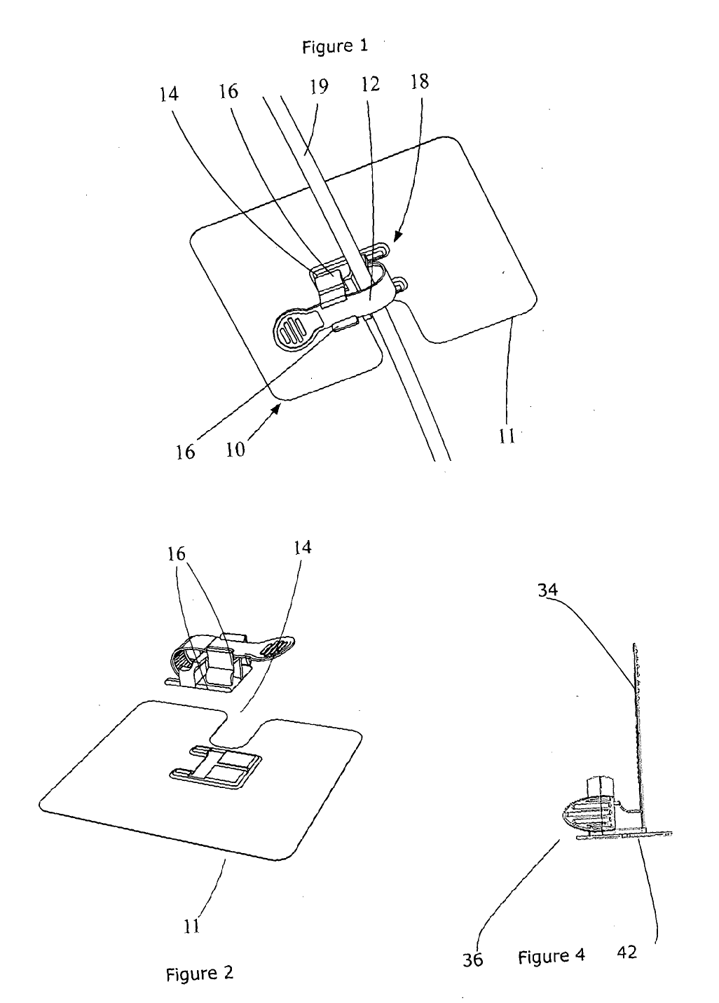

FIG. 1 shows a first medical device securement system in accordance with the invention 10 and comprises an elongate tie member 12 which depends from a base element 14 and which is releasably and adjustably fastened via a jaw system 16 of the base element about an loop 18, through which loop a medical line or device 19 can be fastened. FIG. 2 illustrates the base element 14 separated from an adhesive skin contacting patch member 11, which directly anchors the system 10 to a patient, rather than holding a point in the medical line upstream of the catheter 19 (e.g., the connector between the catheter and the fluid supply tube), for example.

The patch member 11 desirably has a sufficient thickness and tackiness to either secure the medical line or device in order to inhibit relative movement between the securement device 10 and medical line or device 19. The adhesive can be applied to the securement system in any of a variety of conventional ways, such as by transfer technology (e.g., thin film transfer tape) or by sputtering. For example a transfer adhesive available from Minnesota Mining and Manufacturing Company (3M), could be employed. Preferably, the patch member 11 comprises a laminate structure with an upper foam layer (e.g., closed-cell polyethylene foam), and a bottom adhesive layer. The adhesive preferably is a medical-grade adhesive and can be either diaphoretic or nondiaphoretic, depending upon the particular application. The patch member 11 desirably is formed from a polyethylene foam tape (1-2 mm thick) with an acrylic adhesive, 40 to 120 grams/square meter thick.

A removable paper or plastic backing or liner (not shown) suitably covers the bottom adhesive surface before use. This release layer preferably resists tearing and is divided into a plurality of pieces to ease attachment of the pad to a patient's skin. In the illustrated embodiment, the backing is a polycoated, siliconized paper. The backing desirably is conveniently split along a centre line of the flexible contact patch member in order to expose only half of the adhesive bottom surface at one time. The backing liner also advantageously extends beyond at least one edge of the contact patch member, to facilitate removal of the backing liner from the adhesive layer. Although not illustrated, the patch member 11 can include suture holes in addition to the adhesive layer to further secure the anchor pad to the patient's skin.

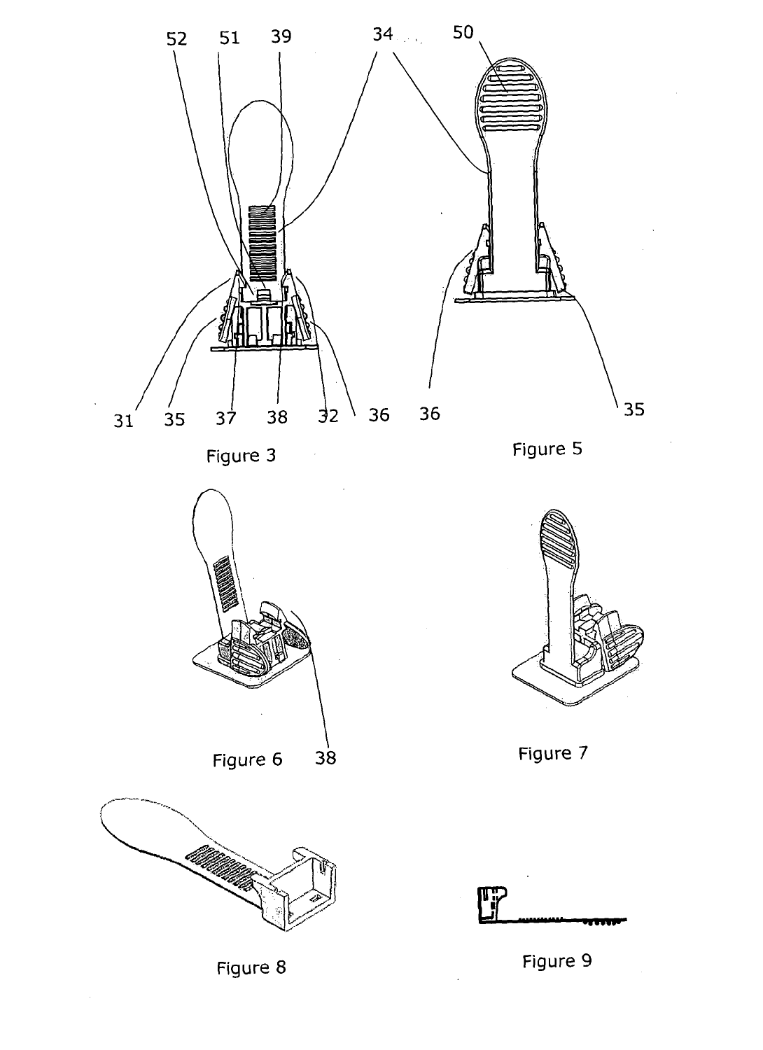

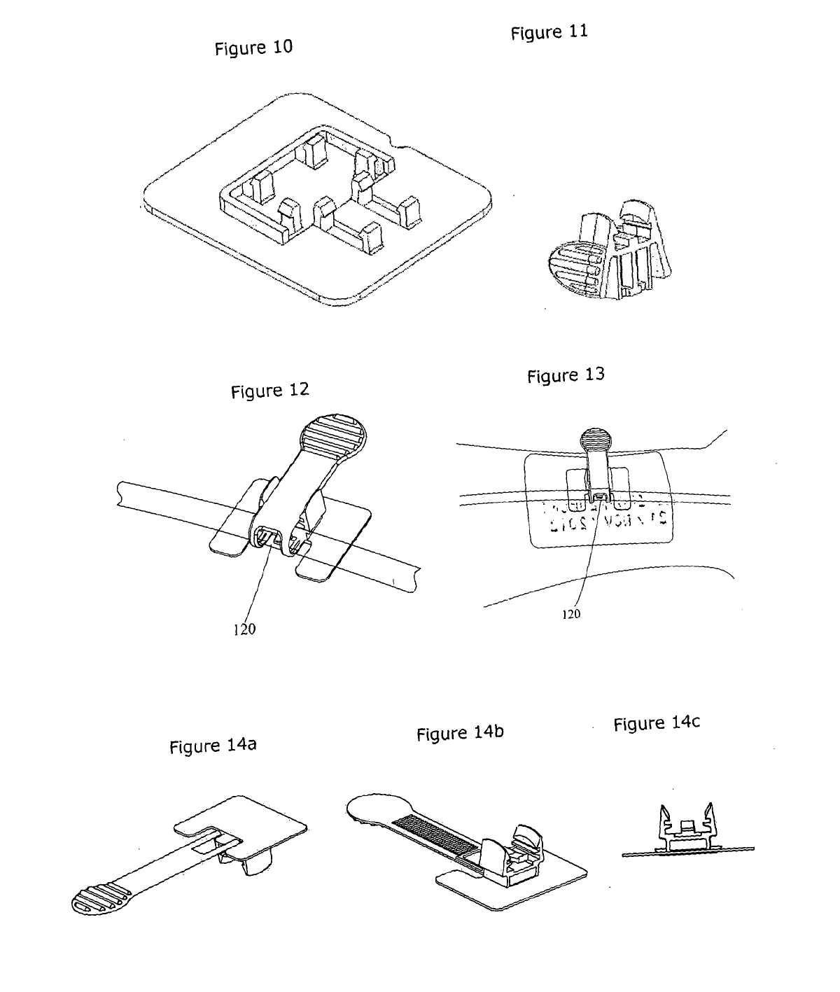

Referring now to FIGS. 3-7, there are shown several views of the securement device to be mounted upon an adhesive patch (not shown for simplicity). As seen, especially from FIG. 3, Jaws 31, 32 define between themselves a passageway of width W, which width also corresponds to a width of tail portion 34, which is shown upstanding perpendicularly to the span between the jaws, prior to fastening about any medical line or device. The Jaws depend from the base and pivot thereabout, having finger paddles 35, 36 depending oppositely with regard to the jaws. The paddles may be provided with grips-like members, as shown by parallel ridges in the diagrams (not referenced) but examples without such ridges have be found to be equally useful and simple to grip by opposing fingers or finger and thumb. Pressing the paddles together widens the gap between the jaws. This can be used to enable the tail to be inserted therebetween and, similarly, to allow release thereof. With reference to FIG. 4, there is shown a saddle 42 for placement of a medical line or feature of a medical device. When a medical line, for example is to be secured, then the distal portion of the tail is urged downwardly also lowering the tail, simultaneously and then, either by pressing the jaws apart by use of angled entrance surfaces 37, 38, or by operation of the finger paddles 35, 36 the tail portion 34 can be located within the jaws 31, 32, to provide a closed loop about the article to be secured. Ratchet member 51 can then locate vis-à-vis the indentations of rack 39 on an inside surface of the tail to prevent any loosening of the lop; by pulling on the tail by its distal end from the jaws will cause the loop to reduce in size, to grip about a device to be secured. A further beneficial feature is that of a securement ridge 52 situated within the jaw assembly, which supports the ratchet pawl 51 operable to engage with respect to ridges 39. Whilst this need not be upstanding—and could compromise a generally planar member—it is conveniently arranged as a ridge axial to the tail such that upon depression of one of the finger paddles 35, or 36, the tail can be twisted so that one side of the tail can depend into the region to the side of the ridge 52 and below the inside edge of jaw 31. or 32. As will be appreciated, this can be of considerable benefit in use, where a connection can be made singlehandedly, reducing the number of staff in attendance with respect to a patient or enable a procedure to be completed more efficiently.

FIG. 5 shows how the tail is equipped with a finger grip portion 50 upon the outer face, at the distal end thereof. FIGS. 6 and 7 show the device, in perspective view, prior to attachment about a medical line or device and prior to attachment to a patch member 11. Sloped entry portion to jaw 32 is shown in detail in FIG. 6

FIGS. 8 through to 11 show the tail 12, base 14 and jaw 16 assembly of the device prior to assembly. It can be seen that the components are few in number, each being formed as separate elements and can be connected together without the aid of adhesive or welding by use of detent clips: see detent teeth arising from the base element, which locate into corresponding features in the underside portions of the tail moulding and jaw moulding 110. Alternatively, the base 14 is secured to the patch member 11 by a solvent bond adhesive, such as cyanoacrylate or other bonding material. One such adhesive is available commercially as from 3M. It will also be appreciated that the tail, base and jaw assembly may be manufactured as a single moulded plastics item. It will be appreciated that simple variations of the inventive system can be devised. For example the rack may comprise edge features of the tail which bear against the jaws 32, 34, which may each—or just one of them—possess a detent member whereby to achieve the simple system of adjustment and release.

The tail 12, base 14 and the jaws 16 of the system 10 can be integrally formed as a unitary piece. This can be accomplished in any of a variety of ways well known to one of skill in the art. Suitable plastics from which the base 14, tail and jaws can be made include, for example, but without limitation, polypropylene, polyethylene, and the like. In particular, acceptable materials include low density polyethylene (LDPE) which is commonly available commercially. When fastened to the adhesive member, the tail 12, base 14 and the jaws 16 could alternatively be introduced as a single part through an aperture in the adhesive patch, and be attached securely with the adhesive on the underside of the adhesive patch.

In use, the health care professional removes the securement device from its protective, sterile packaging (not shown) and peels off the release layer from the adhesive member if provided separate from the packaging. Otherwise, the adhesive member emerges uncovered from the packaging. The health care professional then aligns the catheter or medical device within the loop defined by the saddle and tail; the tail is placed within the gap defined between the jaws of the base; the jaws close and the tail is pulled whereby to secure the line or device. In order to release, the finger paddles are pressed together whereby the jaws open, to thereby release the line or device.

FIGS. 12 and 13 shown a further example of the invention, as shown in perspective view, respectively upon a surface and when fitted to a patient. Reference numeral 120 indicates an aperture in the proximal end of the tail, which enables a health care professional to visibly check security of the tie. By having two strap either side of the aperture in the tail, this also focuses the forces of constriction upon the medical line, to improve grip of said line. As will be appreciated, by having the tail being fastened in a ratchet fashion, the line securement device can be readily tightened. By the same token, if it is necessary to re-route the medical line, it is a simple matter to release the medical line, which adds enormously to the benefits provided by the present invention: not only is it a benefit of the device that the securement device fits a variety of tubing sizes but is can also be readily released once placed upon a patient. It will also be noted that the exit angle of the tail form the saddle is horizontal, not vertical as clearly shown in FIG. 4. This is also clear form FIG. 14b, which together. with FIG. 14b show a further embodiment, where the line securement device is manufactured in a unitary fashion.

The securement device provides a sterile, stable, efficient way to anchor a medical line or article to a patient. The securement device is quickly and easily applied to the medical line or article by using the present invention, the base of which is equally quickly and easily applied to the patient's skin.

In use, a health care provider loops the IV supply tubing around from the insertion site and inserts a portion of the tubing over the seat of the tube clip. The looped section has sufficient slack to form a conventional safety loop. The safety loop absorbs any tension applied to the fluid supply tube to inhibit pulling of the catheter.

The present invention has been disclosed in the context of an exemplary intravenous (IV) catheter. The principles of the present invention, however, are not limited to IV catheters. Instead, it will be understood by one of skill in this art, in view of the present disclosure, that the securement devices and retainers disclosed herein also can be successfully utilized in connection with other types of medical lines, including tubes for fluid communication and electrical wires. For example, but without limitation, the retainers disclosed herein can be adapted to retain CVCs, PICCs, Foley catheters, and haemodialysis catheters, surgical drainage tubes, feeding tubes, chest tubes, nasogastric tubes, scopes, as well as with electrical wires or cables connected to external or implanted electronic devices or sensors.

One skilled in the art may also find additional applications for the devices and devices disclosed herein. Thus, the illustrations and descriptions of securement devices disclosed herein in connection with a catheter are merely exemplary of one possible application of the device. The distal end of the tail can be made with a smoother end; the tail can be of several lengths; indeed, once in use, to would be possible to shorten the length of the tail, although this would reduce ease of removal and adjustment and care would need to be taken to ensure other, adjacent lines are not cut.

1-14. (canceled)

15. A line securement device comprising a base, a securement tail and a lock;
wherein the base has a first face to which an adhesive is applied and a second face to which the securement tail and lock are connected;
wherein the securement tail extends from the base at a proximal end, along a length to a distal end, the tail having a substantially uniform section and having a detent rack upon an engagement portion of the tail;
wherein the lock extends from the base and defines a jaw assembly, directed in a direction from the base and a ratchet, wherein the jaws in a first, rest position, provides a width for passage of said section of said securement tail whilst the ratchet can engage with the teeth of the detent rack to allow movement of the securement tail in a first direction and to prevent movement in a direction opposite to the first direction, at least one jaw member being operable to move such as to allow passage of a section of the securement tail between the jaws.

16. A securement device according to claim 1, wherein the base is attached to an adhesive skin contacting patch member, which directly anchors the system to a patient.

17. A securement device according to claim 2, wherein the patch member comprises a laminate structure with an upper foam layer and a bottom adhesive layer.

18. A securement device according to claim 1, wherein the jaws define between themselves a passageway of width W, which width also corresponds to a width of the securement tail.

19. A securement device according to claim 1, wherein the jaws are provided with finger paddles, operable to cause one or both of the jaws to move outwardly, whereby to widen the gap between the jaws.

20. A securement device according to claim 1, wherein the jaws are provided with sloped entry portions to enable the jaws to open upon presentation of the tail against said sloped portions.

21. A securement device according to claim 1, wherein the tail, has an aperture, about a proximal end whereby to enable visibility of the clamping action about a medical line to be secured.

22. A securement device according to claim 1, wherein the tail securement ratchet-detent means is associated with a central portion of the tail.

23. A securement device according to claim 1, wherein the tail securement ratchet-detent means is associated with at least one edge of the tail and a corresponding at least one jaw.

24. A securement device according to claim 1, wherein the tail, base and jaws are formed as separate elements.

25. A securement device according to claim 10, wherein the component parts can be secured together by one or more of the following: adhesives, heat welding and or clip-detent means.

26. A securement device according to claim 1, wherein the tail, base and jaws are integrally formed as a unitary piece.

27. A securement device substantially as described herein with reference to any one or more of the figures as shown in the drawing sheets.

28. The use of a securement device to secure a medical line substantially as described herein with reference to any one or more of the figures as shown in the drawing sheets.
